(19) United States
(12) Patent Application Publication
(10) Pub. No.: US 2020/0093715 A1
(43) Pub. Date: Mar. 26, 2020

(54) DENTIFRICE COMPOSITIONS FOR TREATMENT OF DENTAL BIOFILM

(71) Applicant: The Procter & Gamble Company, Cincinnati, OH (US)

(72) Inventors: Swapna BASA, Beijing (CN); Yunming SHI, Beijing (CN); Ross STRAND, Singapore (SG)

(21) Appl. No.: 16/530,320

(22) Filed: Aug. 2, 2019

Related U.S. Application Data
(63) Continuation of application No. PCT/CN2017/075532, filed on Mar. 3, 2017.

(51) Int. Cl. A61K 8/24; A61K 8/84; A61K 8/36; A61Q 11/00; A61K 8/34; A61K 8/73

(52) U.S. Cl. CPC A61K 8/24; A61K 8/84; A61K 8/361; A61K 2800/28; A61K 8/345; A61K 8/731; A61K 2800/48; A61Q 11/00

(57) ABSTRACT

Certain alkaline dentifrice compositions with relatively high level of water and calcium-containing abrasive, a bicarbonate salt, are effective in treating dental plaque biofilm.

### FIELD OF THE INVENTION

The present invention relates to dentifrice compositions having improved efficacy to help inhibit biofilm formation or help disrupt biofilm.

### BACKGROUND OF THE INVENTION

Dental plaque (also known as dental biofilm) is a sticky, colorless deposit of bacteria that is constantly forming on the tooth surface. Dental plaque is generally made up of bacteria and extracellular polymer substances (so called “EPS”). EPS are biopolymers of microbial origin in which biofilm microorganisms are embedded. J. Bacteriol. 2007, 189(22):7945. Saliva, food and fluids combine to produce these deposits that collect where the teeth and gums meet. Plaque buildup is the primary factor in poor oral health that can lead to caries and periodontal (gum) disease, including gingivitis. One way dentifrice compositions help prevent and control plaque is by leveraging anti-bacterial agents; however, the disadvantage and formulation challenge is the unintended reactivity of anti-bacterial agents with formulation ingredients and environment of containing calcium carbonate matrix. This may include oxidative degradation, hydrolysis, adsorption or precipitation of oxy-hydroxide species, any of which can impact the bio-availability of the anti-bacterial agent. There is a continuing need to provide such formulations that help prevent plaque formation on teeth and/or minimize the use of antimicrobial agents, particularly in high water and high carbonate dentifrice formulation chassis.

One solution to help inhibit biofilm formation or help disrupt biofilm is the use of Baking Soda (i.e., sodium bicarbonate). Baking soda mechanism of action against biofilm is likely at least two fold. Baking soda can displace calcium ions so as to help disrupt or reduce the biofilm. Calcium ions act as a “glue” or “scaffold” of EPS components of dental biofilm. Baking soda may also act as an abrasive. Some levels of baking soda in dentifrice are reported at over 30 wt %, and sometimes higher than 50 wt %. However, some users report an unpleasant taste experience (attributable to the relatively high level o baking soda). Yet furthermore, dental plaque is particularly problematic in developing markets. And thus, dentifrice solutions are best cost effective (e.g., containing a relatively high level of water). Accordingly, there is a need for baking soda containing dentifrice compositions maxing bicarbonate salt associated therapeutic benefits while minimizing the level of bicarbonate salt (e.g., <26 wt %) as to help with the flavor profile of the composition. There is also a need for a composition that is cost effective for developing markets where arguably the need for such compositions are greatest.

### SUMMARY OF THE INVENTION

The present invention is based on the surprising discovery that the oral care compositions of the present invention comprise relatively low levels of baking soda, but yet are better than some commercialized baking soda containing toothpastes at inhibiting dental biofilm formation or helping disrupt dental biofilm.

An advantage of the present invention is the binding of calcium ions for anti-dental plaque benefits while minimizing any demineralization from the tooth surface.

An advantage of the present invention is improved flavor experience of the present invention as compared to at least some commercialized baking soda containing toothpastes, especially those containing relatively high levels of baking soda.

An advantage of the present invention is mitigating the growth or presence of bacteria that contribute to dental biofilm formation.

An advantage of the present invention is the relatively cost effectiveness of the formulation by relatively high level of water and minimizing other ingredients (such as humectants).

An advantage of the present invention is a phase stable formulation.

One aspect of the invention provides for a dentifrice composition comprising: 30% to 55%, by weight of the composition, of water; 25% to 50%, by weight of the composition, of a calcium-containing abrasive; 1% to 25%, by weight of the composition, of a bicarbonate salt; 0.0025% to 2%, by weight of the composition, of a fluoride ion source; 0% to 2%, by weight of the composition, of a humectant, wherein the humectant is selected from sorbitol, glycerol, or and a combination thereof; and wherein said composition has a pH greater than 7.8.

Yet another aspect of the invention provides a method of treating dental biofilm comprising the step of brushing teeth with a composition of the present invention.

Yet still another aspect of the invention provides a method preventing or mitigating plaque formation on tooth enamel comprising the step of brushing teeth with a dentifrice composition of the present invention.

These and other features, aspects, and advantages of the present invention will become evident to those skilled in the art from the detailed description which follows.

### BRIEF DESCRIPTION OF THE DRAWINGS

FIG. 1 is a perspective view of an oral splint with Hydroxyapatite (“HA”) disks attached thereto.

### DETAILED DESCRIPTION OF THE INVENTION

### Definitions

The term “comprising” as used herein means that steps and ingredients other than those specifically mentioned can be added. This term encompasses the terms “consisting of” and “consisting essentially of.” The compositions of the present invention can comprise, consist of, and consist essentially of the essential elements and limitations of the invention described herein, as well as any of the additional or optional ingredients, components, steps, or limitations described herein.

The term “dentifrice” as used herein means paste, gel, powder, tablets, or liquid formulations, unless otherwise specified, that are used to clean the surfaces of the oral cavity. Preferably the dentifrice compositions of the present invention are single phase compositions. One example of a dentifrice is toothpaste (for brushing teeth). The term “teeth” as used herein refers to natural teeth as well as artificial teeth or dental prosthesis.

All percentages, parts and ratios are based upon the total weight of the compositions of the present invention, unless otherwise specified. All such weights as they pertain to listed ingredients are based on the active level and, therefore do not include solvents or by-products that may be included in commercially available materials, unless otherwise specified. The term “weight percent” may be denoted as “wt %” herein. All molecular weights as used herein are weight average molecular weights expressed as grams/mole, unless otherwise specified.

As used herein, the articles including “a” and “an” when used in a claim, are understood to mean one or more of what is claimed or described.

As used herein, the words “preferred”, “preferably” and variants refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

### Water

The dentifrice compositions of the present invention comprise herein from 30% to 55%, by weight of the composition, of water. For example, the dentifrice composition may comprise 34%, 38%, 40%, 42%, 44%, 46%, 48%, or 50%, by weight of the composition, of water. Preferably, the dentifrice composition comprises from 30% to 55%, more preferably from 34% to 55%, yet more preferably from 35% to 55%, yet still more preferably from 40% to 55%, by weight of the composition, of water. The water may be added to the formulation and/or may come into the composition from the inclusion of other ingredients. Preferably the water is USP water.

### Calcium-Containing Abrasive

The compositions of the present invention comprise from 25% to 50%, by weight of the composition, of a calcium-containing abrasive, wherein preferably the calcium-containing abrasive is selected from the group consisting of calcium carbonate, calcium glycerophosphate, dicalcium phosphate, tricalcium phosphate, calcium orthophosphate, calcium metaphosphate, calcium polyphosphate, calcium oxyapatite, sodium carbonate, and combinations thereof; wherein more preferably the calcium-containing abrasive is calcium carbonate. Preferably, the composition comprises from 27% to 47%, more preferably from 27% to 37%, even more preferably from 28% to 34%, by weight of the composition, alternatively combinations thereof, of a calcium-containing abrasive.

Preferably, the calcium-containing abrasive is calcium carbonate. More preferably, the calcium-containing abrasive is selected from the group consisting of fine ground natural chalk, ground calcium carbonate, precipitated calcium carbonate, and combinations thereof. Non-limiting examples of the weight percentages of the calcium-containing abrasive include: 27%, 28%, 29%, 30%, 31%, 32%, 33%, 34%, 35%, 36%, or 37%, by weight of the composition, preferably wherein the calcium-containing abrasive is calcium carbonate.

Fine ground natural chalk (FGNC) is one of the more preferred calcium-containing abrasives useful in the present invention. It is obtained from limestone or marble. FGNC may also be modified chemically or physically by coating during milling or after milling by heat treatment. Typical coating materials include magnesium stearate or oleate. The morphology of FGNC may also be modified during the milling process by using different milling techniques, for example, ball milling, air-classifier milling or spiral jet milling. One example of natural chalk is described in WO 03/030850 having a medium particle size of 1 to 15 μm and a BET surface area of 0.5 to 3 m²/g. The natural calcium carbonate may have a particle size of 325 to 800 mesh, alternatively a mesh selected from 325, 400 600, 800, or combinations thereof; alternatively, the particle size is from 0.1 to 30 microns, or from 0.1 to 20 microns, or from 5 to 20 microns. In one embodiment, the composition comprises from 0% to 5%, preferably 0% to 3%, more preferably 0% to 1%, by weight of the composition, of a silicate; yet more preferably the composition is substantially free silicate.

### Bicarbonate Salt

The compositions of the present invention comprise from 1% to 30%, by weight of the composition, of a bicarbonate salt, wherein preferably the bicarbonate salt is selected from sodium bicarbonate or calcium bicarbonate, more preferably sodium bicarbonate. Preferably the compositions comprise 1%, 2%, 3%, 4%, 5%, 6%, 7%, 8%, 9%, 10%, 11%, 12%, or 13%; more preferably from 1% to 25%, yet more preferably from 1% to 20%, yet still more preferably from 1% to 15%, yet still more preferably from 1% to 10%, alternatively from 3% to 9%, alternatively combinations thereof, by weight of the composition, of the bicarbonate salt (preferably sodium bicarbonate).

### Fluoride Ion Source

The compositions may include an effective amount of an anti-caries agent. In one example, the anti-caries agent is a fluoride ion source. The fluoride ion may be present in an amount sufficient to give a fluoride ion concentration in the composition at 25° C., and/or in one embodiment can be used at levels of from 0.0025% to 5% by weight of the composition, alternatively from 0.005% to 2.0% by weight of the composition, to provide anti-caries effectiveness. Representative fluoride ion sources include: stannous fluoride, sodium fluoride, potassium fluoride, amine fluoride, sodium monofluorophosphate, and zinc fluoride. In one example the dentifrice composition contains a fluoride source selected from stannous fluoride, sodium fluoride, and combinations thereof. In one embodiment, the fluoride ion source is sodium monofluorophosphate, and wherein the composition comprises 0.0025% to 2%, by weight of the composition, of the sodium monofluorophosphate, alternatively from 0.5% to 1.5%, alternatively from 0.6% to 1.7%, alternatively combinations thereof. In another example, the composition comprises from 0.0025% to 2%, by weight of the composition, of a fluoride ion source. In one example, the dentifrice compositions of the present invention may have a dual fluoride ion source, specifically sodium monofluorophosphate and an alkaline metal fluoride. Without wishing to be bound by theory, such an approach may provide an improvement in mean fluoride uptake.

pH

The pH of the dentifrice composition may be greater than pH 7.8, preferably greater than pH 8, more preferably from greater than pH 8.0 to pH 11, yet more preferably from pH 8.5 to pH 11, yet still more preferably at or greater than pH 9 to pH 10.5. Alternatively the pH is from pH 9 to pH 10. The relatively high pH of the present inventive composition is for fluoride stability. Without wishing to be bound theory, at below pH 8 calcium ion may bind with the fluoride. Thus, it is desirable to have the dentifrice composition have a greater than pH 8.0 to maximize the stability of the fluoride ion source. A method for assessing pH of dentifrice is described is below. For purposes of clarification, although the analytical method describes testing the dentifrice composition when freshly prepared, for purposes of claiming the present invention, the pH may be taken at anytime during the product's reasonable lifecycle (including but not limited to the time the product is purchased from a store and brought to the user's home).

### pH Modifying Agent

The dentifrice compositions herein may include an effective amount of a pH modifying agent, alternatively wherein the pH modifying agent is a pH buffering agent. The pH modifying agents, as used herein, refer to agents that can be used to adjust the pH of the dentifrice compositions to the above-identified pH range. The pH modifying agents may include alkali metal hydroxides, ammonium hydroxide, organic ammonium compounds, carbonates, sesquicarbonates, borates, silicates, phosphates, imidazole, and mixtures thereof. Specific pH agents include monosodium phosphate (monobasic sodium phosphate or “MSP”), trisodium phosphate (sodium phosphate tribasic dodecahydrate or “TSP”), sodium benzoate, benzoic acid, sodium hydroxide, potassium hydroxide, alkali metal carbonate salts, sodium carbonate, imidazole, pyrophosphate salts, sodium gluconate, lactic acid, sodium lactate, citric acid, sodium citrate, phosphoric acid. In one embodiment, 0.01% to 3%, preferably from 0.1% to 1%, by weight of the composition, of TSP, and 0.001% to 2%, preferably from 0.01% to 0.3%, by weight of the composition, of monosodium phosphate is used. Without wishing to be bound by theory, TSP and monosodium phosphate may also have calcium ion chelating activity and therefore provide some monofluorophosphate stabilization (in those formulations containing monofluorophosphate).

A method for assessing pH of dentifrice is described. The pH is measured by a pH Meter with Automatic Temperature Compensating (ATC) probe. The pH Meter is capable of reading to 0.001 pH unit. The pH electrode may be selected from one of the following (i) Orion Ross Sure-Flow combination: Glass body—VWR #34104-834/Orion #8172BN or VWR #10010-772/Orion #8172BNWP; Epoxy body—VWR #34104-830/Orion #8165BN or VWR #10010-770/Orion #8165BNWP; Semi-micro, epoxy body—VWR #34104-837/Orion #8175BN or VWR #10010-774/Orion #3175BNWP; or (ii) Orion PerpHect combination: VWR #34104-843/Orion #8203BN semi-micro, glass body; or (iii) suitable equivalent. The automatic temperature compensating probe is Fisher Scientific, Cat #13-620-16.

A 25% by weight slurry of dentifrice is prepared with deionized water, and thereafter is centrifuged for 10 minutes at 15000 rotations-per-minute using a SORVALL RC 28S centrifuge and SS-34 rotor (or equivalent gravitational force, at 24149 g force). The pH is assessed in supernatant after one minute or the taking reading is stabilized. After each pH assessment, the electrode is washed with deionized water. Any excess water is wiped with a laboratory grade tissue. When not in issue, the electrode is kept immersed in a pH 7 buffer solution or an appropriate electrode storage solution.

### Low Level or Free of Humectants

The compositions herein contains relatively low amount, or even be substantially free or free, of humectants. Non-limiting examples of humectant levels, by weight of the oral care composition, include 0.1%, 0.5%, 1%, 1.5%, 2%, or 0%. Without wishing to be bound by theory, the presence of humectant (e.g., sorbitol/glyercol) may have a negative role in fluoride uptake in dental plaque in the high water and high carbonate containing dentifrice formulations of the present invention. Reduced levels of sorbitol/glycerol in these dentifrice compositions provide superior fluoride uptake results. Preferably the dentifrice compositions of the present invention comprise from 0% to 2%, by weight of the composition, of a humectant, wherein the humectant is selected from soribitol, glycerol, and combination thereof; more preferably the composition contain from 0% to 1.5%, yet more preferably 0% to 1%, yet still more preferably 0% to 0.5%, by weight of the composition of said humectant; alternatively from 0% to 0.1%; alternatively, the composition is substantially free of the subject humectant.

The term “humectant,” for the purposes of present invention, include edible polyhydric alcohols such as glycerin, sorbitol, xylitol, butylene glycol, propylene glycol, and combinations thereof. In one example, the humectant is a polyol, preferably wherein the polyol is selected from sorbitol, glycerin, and combinations thereof. In yet another example, the humectant is sorbitol. A potential advantage of having a dentifrice composition that contains low levels of humectant (i.e., at or less than 2 wt %), without wishing to be bound by theory, is those dentifrice compositions that are free of humectants such as glyercol or sorbitol may provide better fluoride uptake as compared to those compositions having the high levels of such humectants. In one example, the dentifrice compositions of the present invention comprise from 0% to 2%, preferably 0% to 1%, more preferably 0% to 0.5%, by weight of the composition, of glycerin, sorbitol; alternatively from 0 wt % to 0.1 wt %, or combinations thereof; yet more preferably the composition is substantially free of both glycerin and sorbitol.

### Thickening System

The dentifrice compositions of the present invention may comprise a thickening system. Preferably the dentifrice composition comprises from 0.5% to 4%, preferably from 0.8% to 3.5%, more preferably from 1% to 3%, yet still more preferably from 1.3% to 2.6%, by weight of the composition, of the thickening system. More preferably the thickening system comprises a thickening polymer, a thickening silica, or a combination thereof. Yet more preferably, when the thickening system comprises a thickening polymer, the thickening polymer is selected from a carboxymethyl cellulose, a linear sulfated polysaccharide, a natural gum, and combination thereof. Yet still more preferably, when the thickening system comprises a thickening polymer, the thickening polymer is selected from the group consisting of: (a) 0.01% to 3% of a carboxymethyl cellulose (“CMC”) by weight of the composition, preferably 0.1% to 2.5%, more preferably 0.2% to 1.5%, by weight of the composition, of CMC; (b) 0.01% to 2.5%, preferably 0.05% to 2%, more preferably 0.1% to 1.5%, by weight of the composition, of a linear sulfated polysaccharide, preferably wherein the linear sulfated polysaccharide is a carrageenan; (c) 0.01% to 7%, preferably 0.1% to 4%, more preferably from 0.1% to 2%, yet more preferably from 0.2% to 1.8%, by weight of the composition, of a natural gum; (d) combinations thereof. Preferably when the thickening system comprises a thickening silica, the thickening silica is from 0.01% to 10%, more preferably from 0.1% to 9%, yet more preferably 1% to 8% by weight of the composition.

Preferably the linear sulfated polysaccharide is a carrageenan (also known as carrageenin). Examples of carrageenan include Kappa-carrageenan, Iota-carrageenan, Lambda-carrageenan, and combinations thereof.

In one example the thickening silica is obtained from sodium silicate solution by destabilizing with acid as to yield very fine particles. One commercially available example is ZEODENT® branded silicas from Huber Engineered Materials (e.g., ZEODENT® 103, 124, 113 115, 163, 165, 167).

In one example the CMC is prepared from cellulose by treatment with alkali and monochloro-acetic acid or its sodium salt. Different varieties are commercially characterized by viscosity. One commercially available example is Aqualon™ branded CMC from Ashland Special Ingredients (e.g., Aqualon™ 7H3SF; Aqualon™ 9M3SF Aqualon™ TM9A; Aqualon™ TM12A).

Preferably a natural gum is selected from the group consisting of gum karaya, gum arabic (also known as acacia gum), gum tragacanth, xanthan gum, and combination thereof. More preferably the natural gum is xanthan gum. Xanthan gum is a polysaccharide secreted by the bacterium Xanthomonas camestris. Generally, xanthan gum is composed of a pentasaccharide repeat units, comprising glucose, mannose, and glucuronic acid in a molar ratio of 2:2:1, respectively. The chemical formula (of the monomer) is C₃₅H₄₉O₂₉. In one example, the xanthan gum is from CP Kelco Inc (Okmulgee, US).

### Viscosity

Preferably the dentifrice compositions of the present invention have a viscosity range from 150,000 centipoise to 850,000 centipoise (“cP”). A method for assessing viscosity is described. The viscometer is Brookfield® viscometer, Model DV-I Prime with a Brookfield “Helipath” stand. The viscometer is placed on the Helipath stand and leveled via spirit levels. The E spindle is attached, and the viscometer is set to 2.5 RPM. Detach the spindle, zero the viscometer and install the E spindle. Then, lower the spindle until the crosspiece is partially submerged in the paste before starting the measurement. Simultaneously turn on the power switch on the viscometer and the helipath to start rotation of the spindle downward. Set a timer for 48 seconds and turn the timer on at the same time as the motor and helipath. Take a reading after the 48 seconds. The reading is in cP.

### PEG

The compositions of the present invention may optionally, but preferably, comprise polyethylene glycol (PEG), of various weight percentages of the composition as well as various ranges of average molecular weights. In one aspect of the invention, the compositions have from 0.1% to 5%, preferably from 0.5% to 4%, more preferably from 1% to 3%, by weight of the composition, of PEG. In another aspect of the invention, the PEG is one having a range of average molecular weight from 100 Daltons to 1600 Daltons, preferably from 200 to 1000, alternatively from 400 to 800, alternatively from 500 to 700 Daltons, alternatively combinations thereof. PEG is a water soluble linear polymer formed by the addition reaction of ethylene oxide to an ethylene glycol equivalent having the general formula is: H—(OCH₂CH₂)ₙ—OH. One supplier of PEG is Dow Chemical Company under the brandname of CARBOWAX™. Without wishing to be bound by theory, having some PEG in the dentifrice composition may help with physical stability.

### Sweetener

The oral care compositions herein may include a sweetening agent. These sweetening agents may include saccharin, dextrose, sucrose, lactose, maltose, levulose, aspartame, sodium cyclamate, D-tryptophan, dihydrochalcones, acesulfame, sucralose, neotame, and mixtures thereof. Sweetening agents are generally used in oral compositions at levels of from 0.005% to 5%, by weight of the composition, alternatively 0.01% to 1%, alternatively from 0.1% to 0.5%, alternatively combinations thereof.

### Anti-Calculus Agent

The dentifrice compositions may include an effective amount of an anti-calculus agent, which in one embodiment may be present from 0.05% to 50%, by weight of the composition, alternatively from 0.05% to 25%, alternatively from 0.1% to 15% by weight of the composition. Non-limiting examples include those described in US 2011/0104081 A1 at paragraph 64, and those described in US 2012/0014883 A1 at paragraphs 63 to 68, as well as the references cited therein. One example is a pyrophosphate salt as a source of pyrophosphate ion. In one embodiment, the composition comprises tetrasodium pyrophosphate (TSPP) or disodium pyrophosphate or combinations thereof, preferably 0.01% to 2%, more preferably from 0.1% to 1%, by weight of the composition, of the pyrophosphate salt. Without wishing to be bound by theory, TSPP may provide not only calcium chelating thereby mitigating plaque formation, but may also provide the additional benefit of monofluorophosphate stabilization (in those formulations containing monofluorophosphate).

### Surfactant

The dentifrice compositions herein may include a surfactant. The surfactant may be selected from anionic, nonionic, amphoteric, zwitterionic, cationic surfactants, or mixtures thereof. The composition may include a surfactant at a level of from 0.01% to 10%, from 0.025% to 9%, from 0.05% to 5%, from 0.1% to 2.5%, from 0.5% to 2%, or from 0.1% to 1% by weight of the total composition. Non-limiting examples of anionic surfactants may include those described at US 2012/0082630 A1 at paragraphs 32, 33, 34, and 35. Non-limiting examples of zwitterionic or amphoteric surfactants may include those described at US 2012/0082630 A1 at paragraph 36; cationic surfactants may include those described at paragraphs 37 of the reference; and nonionic surfactants may include those described at paragraph 38 of the reference. In one embodiment the composition comprises 0.1% to 5%, preferably 0.1% to 3%, alternatively from 0.3% to 3%, alternatively from 1.2% to 2.4%, alternatively from 1.2% to 1.8%, alternatively from 1.5% to 1.8%, by weight of the composition, alternatively combinations thereof, of the anionic surfactant sodium lauryl sulfate (SLS).

### Colorant

The compositions herein may include a colorant. Titanium dioxide is one example of a colorant. Titanium dioxide is a white powder which adds opacity to the compositions. Titanium dioxide generally can comprise from 0.25% to 5%, by weight of the composition.

### Flavorant

The compositions herein may include from 0.001% to 5%, alternatively from 0.01% to 4%, alternatively from 0.1% to 3%, alternatively from 0.5% to 2%, alternatively 1% to 1.5%, alternatively 0.5% to 1%, by weight of the composition, alternatively combinations thereof, of a flavorant composition. The term flavorant composition is used in the broadest sense to include flavor ingredients, or sensates, or sensate agents, or combinations thereof. Flavor ingredients may include those described in US 2012/0082630 A1 at paragraph 39; sensates and sensate agents may include those described at paragraphs 40-45, incorporated herein by reference. Excluded from the definition of flavorant composition is “sweetener” (as described above).

### Examples

Methods are first described, then oral care compositions are provided, and lastly results are discussed. A biofilm is treated by the oral care compositions. These oral care compositions include comparative examples and inventive ones (Table 1). The term “biofilm” refers to the layer(s) of cells attached to a surface. A biofilm can be a bacterial biofilm that includes both alive and growing microbe cells as well as dead microbe cells. The biofilm can be composed of one cell type or it may be composed of two or more cell types, for example, a biofilm complex that is a multispecies bacterial community. A specific type of biofilm is “dental biofilm” (also known as “plaque biofilm,” used herein interchangeably) which is biofilm that typically forms on tooth surfaces in the human mouth). Bacteria in a plaque biofilm have significantly different physiological characteristics, e.g. increased resistance to detergents and antibiotics, making biofilm research highly important. A non-limiting list of oral bacterial species is described at U.S. Pat. No. 6,309,835 B1, column 7, lines 12-30. These adherent microbe cells are frequently embedded within a self-produced matrix of extracellular polymeric substance (“EPS”). EPS are biopolymers of microbial origin in which biofilm microorganisms are embedded. J. Bacteriol. 2007. 189(22):7945. Biofilm extracellular polymeric substance is a polymeric conglomeration generally composed of calcium, extracellular DNA, proteins, and polysaccharides.

The substrate for dental biofilm growth is described. Hydroxyapatite (HA) disks are used for in situ growth of biofilm. The HA disks are designed having three parallel grooves (300 um wide, 300 um deep for two sides' grooves, while 500 um wide, 500 um deep for the middle groove) in each disk. When attaching disks to subject's mouth, keeping these grooves vertical, to mimic interproximal gap between teeth, the hard-to-clean area where plaque accumulates. This model allows the collection of undisturbed natural grown plaque biofilm from the grooves. HA disks are manufactured by Shanghai Bei'erkang biomedicine limited company.

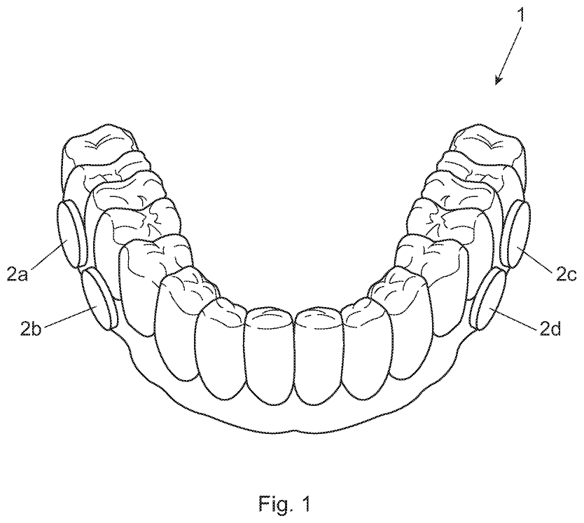

Human subjects wearing a splint are described. Each subject wears up to 12 HA disks on the splint to make sure at least 9 HA disks are available after 48 hours. A non-limiting example of such a splint and HA disks are shown in FIG. 1. The device (1) holds a plurality of HA disks (2a-2d). Although not shown in FIG. 1, the disks can be positioned such that the recede in the inter-dental space between the teeth (since this location is prone to plaque (given the difficulty in cleaning etc.)). The subjects withdraw the splint (the splint stored in an opaque container under humid conditions) only during meals and to perform oral hygiene procedures Immediately thereafter, the splint is worn again. Subjects are asked to use a straw when drinking.

The procedure for in situ biofilm release from HA disk is described. All HA disks are removed from the splint at 48 hours by tweezers. Tweezers are used to hold the edge of HA chips and transfer the HA disk to a 2 ml centrifuge tube containing PBS (phosphate buffered saline) solution. Tweezers are washed thoroughly (water; 75% alcohol; and then deionized water) before every disk transfer.

The preparation for PBS solution is described. One phosphate buffer saline tablet (available from Sigma-Aldrich Corp., MO, USA) is added to 200 grams deionized water in a 250 ml beaker. After stirring thoroughly, the solution is stored at 4° C. for up to 30 days before usage.

The preparation for oral care composition (e.g., toothpaste) supernatant is described. 15 grams of deionized water is added to 5 grams toothpaste in a 100 ml beaker. After stirring thoroughly, the mixture is centrifuge 11,000×g for 20 minutes. The supernatant is prepared immediately before usage or at most one day before usage and stored at 4° C.

After the HA disks are removed from the splint, the disks are used for ex vivo treatment by PBS and/or different oral care compositions. After being treated with PBS and/or the oral care composition (e.g., toothpaste) supernatant and labeled with EPS fluorescent probe and calcium fluorescent probe, the biofilm in the grooves is measured by confocal laser scanning microscopy (CLSM).

Disk preparation is described. The HA disks are rinsed in PBS solution and each HA disk is divided into two halves by tweezers. Thereafter each half-disk specimen is placed into 500-1000 ul of PBS solution statically for 1 minute. Each specimen is treated for two minutes by either PBS solution or a toothpaste supernatant. Each specimen is washed by holding each disk with tweezers, shaken for ten rounds of back and forth in 1 ml of PBS solution. This washing cycle is repeated. Thereafter each specimen is immersed into 500-1000 ul PBS solution statically for 5 minutes.

Fluorescence staining and microscopy is described. Fluorescence labeled calcium probes are molecules that exhibit an increase in fluorescence upon binding Ca2+. Fluo-3™ is used to image the spatial dynamics of Ca2+ signaling. Biofilm may be treated with the AM™ ester forms of calcium probes by adding the dissolved probe directly to biofilm. Fluo-3™, AM™, cell permeant fluorescent probes are used for intracellular and extracellular calcium staining using confocal microscopy, flow cytometry, and microplate screening applications (absorption/emission maxima ˜506/526 nm). It is reported that the Concanavalin ATM (Con A), Alexa Fluor® 594 Conjugate is a reliable alternative to stain EPS of biofilm. Alexa Fluor® 594 conjugate of Con A exhibits the bright, red fluorescence of the Alexa Fluor® 594 dye (absorption/emission maxima ˜590/617 nm). Concanavalin ATM, Alexa Fluor® 594 Conjugate selectively binds to α-mannopyranosyl and α-glucopyranosyl residues which are rich in EPS part of biofilm. After treatment and immersing, each half-disk specimen is stained with a dye mixture solution of the Fluo-3™, AM™, cell permeant fluorescent probe together with Concanavalin ATM, Alexa Fluor® 594 Conjugate probe (containing 5 uM Fluo-3™+5 uM Con-ATM) for 30 minutes in the dark. After staining, each specimen is immersed into 500-1000 ul PBS solution statically for 2 minutes. The specimens are washed again, by holding each disk with tweezers, shaken for five rounds of back and forth in 1 ml PBS solution, and repeated. For Fluo-3/Con-A dye co-stained samples, the following parameters are used: λex=488 nm/561 nm respectively, λem=526/617 nm respectively, 20× objective lens, and scanning from bottom of disk surface bacteria for 60 um depth with step size of 3 um. The other half-disk specimen is stained with L7012 LIVE/DEAD® dye solution (containing 5 uM Syto-9+30 uM propidium iodide) for 15 minutes in the dark for assessing bactericidal efficacy. For the L7012 LIVE/DEAD® dyed stained sample, the following parameters are used: λex=488 nm, λem=500/635 nm respectively, 20× objective lens, and scanning from bottom of surface bacteria for 60 um depth with step size of 3 um.

Confocal Laser Scanning Microscopy (CLSM) is described. The Leica™ TCS SP8 AOBS spectral confocal microscope (available from Leica Mikroskopie GmbH, Wetzlar, Germany) is used. The confocal system consists of a Leica™ DM6000B upright microscope and a Leica™ DMIRE2 inverted microscope. An upright stand is used for applications involving slide-mounted specimens; whereas the inverted stand, having a 37° C. incubation chamber and CO₂ enrichment accessories, provides for live cell applications. The microscopes share an exchangeable laser scan head and, in addition to their own electromotor-driven stages, a galvanometer-driven high precision Z-stage which facilitates rapid imaging in the focal (Z) plane. In addition to epifluorescence, the microscopes support a variety of transmitted light contrast methods including bright field, polarizing light and differential interference contrast, and are equipped with 5×, 20×, 40×, 63× (oil and dry) and 100× (oil) Leica™ objective lenses.

The laser scanning and detection system is described. The TCS SP8 AOBS confocal laser scanning system (available from Leica Lasertechnik GmbH, Heidelberg, Germany) is supplied with four lasers (one diode, one argon, and two helium neon lasers) thus allowing excitation of a broad range of fluorochromes within the UV, visible and far red ranges of the electromagnetic spectrum. The design of the laser scan head, which incorporates acousto-optical tunable filters (AOTF), an acousto-optical beam splitter (AOBS) and four prism spectrophotometer detectors, permits simultaneous excitation and detection of three fluorochromes. The upright microscope also has a transmission light detector making it possible to overlay a transmitted light image upon a fluorescence recording.

Leica™ Confocal software LAS AF3.3.0 is used. The confocal is controlled via a standard Pentium PC equipped with dual monitors and running Leica™ Confocal Software. The Leica Confocal Software LAS AF3.3.0 (available from Leica Lasertechnik GmbH, Heidelberg, Germany) provides an interface for multi-dimensional image series acquisition, processing and analysis, that includes 3D reconstruction and measurement, physiological recording and analysis, time-lapse, fluorochrome co-localization, photo-bleaching techniques such as FRAP and FRET, spectral immixing, and multicolour restoration. Regarding image analysis, for Fluo-3™/Con-ATM stained specimens, both fluorescence channels are chosen to quantify fluorescence intensity ratio of green pixels (Calcium) to red pixels (EPS) and Con-ATM fluorescence channel is chosen to measure the biofilm thickness. The L7012 LIVE/DEAD® dye stained specimens are chosen to assess bacterial vitality by quantifying fluorescence intensity percentage of green pixels (alive bacteria) of all pixels (all bacteria—no matter living or dead).

Table 1 describes four oral care compositions. Generally, examples 1 and 2 are comparative, while examples 3 and 4 are inventive. Example 1 notably does not contain any sodium bicarbonate. Example 2 is also a comparative example acting as a control (compared to Examples 3 and 4) by notably not containing any sodium bicarbonate (and compensating with additional water). Examples 3 and 4 are inventive compositions notably containing sodium bicarbonate. Example 3 has 9 percent by weight of the composition of sodium bicarbonate while Example 4 contains 20 percent of sodium bicarbonate.

TABLE 1
Compositional components of comparative examples 1
and 2, and inventive examples 3 and 4 are provided.

| Components: | Ex 1 | Ex 2 | Ex 3 | Ex 4 |
|---|---|---|---|---|
| (Wt %) | Comparative | Comparative | Inventive | Inventive |
| Sodium Bicarbonate | 0 | 0 | 9 | 20 |
| NaCl | 0.8 | 0 | 0 | 0 |
| KCl | 0.02 | 0 | 0 | 0 |
| Na₂HPO₄ | 0.142 | 0 | 0 | 0 |
| KH₂PO₄ | 0.024 | 0 | 0 | 0 |
| Water | 99.014 | 54.27 | 45.27 | 34.27 |
| CaCO₃ | 0 | 32 | 32 | 32 |
| Sodium Mono- | 0 | 1.10 | 1.10 | 1.10 |
| fluorophosphate | | | | |
| Sodium Caboxy- | 0 | 0.91 | 0.91 | 0.91 |
| methyl Cellulose | | | | |
| Carrageenan | 0 | 1.20 | 1.20 | 1.20 |
| Thickener Silica | 0 | 2.62 | 2.62 | 2.62 |
| Sodium | 0 | 4.00 | 4.00 | 4.00 |
| LaurylSulfate | | | | |
| Flavor | 0 | 1.00 | 1.00 | 1.00 |
| Sodium Mono- | 0 | 0.08 | 0.08 | 0.08 |
| phosphate | | | | |
| Sodium | 0 | 0.42 | 0.42 | 0.42 |
| Triphosphate | | | | |
| Sodium Saccharine | 0 | 0.40 | 0.40 | 0.40 |
| PEG 600 | 0 | 2.00 | 2.00 | 2.00 |
| Total: | 100.0 | 100.00 | 100.00 | 100 |

Data is discussed. In reference to Table 2, the fluorescence intensity ratio of Calcium/EPS within in situ plaque biofilm and average biofilm thickness measured for various examples (1-4) and toothpaste formulations (commercialized examples) are provided. The first column of Table 2 identifies the product. In turn, each product includes the examples described in Table 1 above (namely examples 1-4) as well as two commercialized products examples A and B. Notably examples A and B contain 67 wt % and 9 wt % baking soda, respectively. The second column of Table 2 indicates the amount of baking soda (i.e., sodium carbonate) in each example. The third column indicates the Fluorescence Ratio of Calcium/EPS. Lastly, the fourth column indicates the dental biofilm thickness. The procedures as previously described are used. Briefly, the biofilm is treated with the subject oral care compositions first, and then the treated biofilm is labeled with the EPS and calcium probes. Using software, the mean fluorescence intensities of green pixels (staining calcium ions) and red pixels (staining EPS) are given. The fluorescence intensity ratio of green pixels to red pixels is then calculated. Regarding biofilm thickness assessment, six selected fields of Con-A™ fluorescence channel of each specimen are evaluated. These fields are considered as representative of the whole sample after the observer's general examination. The distance is measured from the surface of the biofilm to its base, measuring the thickness of the field, and subsequently the mean thickness of the biofilm of the corresponding specimen is calculated. The lower the fluorescence ratio, the more effective is the composition. The lower the biofilm thickness, the more effective is the composition.

Still referencing Table 2, example 1 (“Ex 1”) Phosphate Buffer Solution (“PBS”) is used as the negative control. Accordingly, Ex 1 is the least effective (compared to the other compositions) in both as to the fluorescence ratio of Calcium (“Ca”)/EPS and impact on dental biofilm thickness. Example A (“Ex A”) is SENSODYNE® PARODONTAX™ toothpaste (“PARODONTAX”, LOT #14042610, containing 67 wt % sodium bicarbonate), a commercially available toothpaste composition. Ex A shows significantly reduced Fluorescence Ratio of Ca/EPS and biofilm thickness compared to the negative control (i.e., Ex 1). Notable, however, there is no significant difference between Ex A and Example 3 (“Ex 3”). Ex 3 is an inventive composition that contains 9 percent, by weight of the composition, of sodium carbonate. This is considerably less than the 67 wt % of Example A. Example B (“Ex B”) is a CREST® Baking Soda & Peroxide Whitening (LOT: 632232 EXPOCT19); containing around 9 wt % of Baking Soda. Ex B also shows significantly reduced Fluorescence Ratio of Ca/EPS and biofilm thickness compared to the negative control of Ex 1. Notably, however, inventive composition Ex 3, taking into account the error margin, is significantly more effective than Ex B. Ex. 4 is the most effective in reduced Fluorescence Ratio of Ca/EPS and biofilm thickness.

TABLE 2
Data is provided on the fluorescence intensity ratio of Calcium/EPS
in biofilm, and mean biofilm thickness for various comparative
and inventive dentifrice products/formulations.

| | Weight Percentage | Fluorescence Ratio | Biofilm |
|---|---|---|---|
| | of sodium carbonate | of Ca/EPS | Thickness |
| Product¹ | (wt %) | (Mean ± SD) | (um)(Mean ± SD) |
| Ex 1 | 0% | 7.42 ± 0.70 | 31.63 ± 0.52 |
| Ex A¹ | 67% | 4.07 ± 0.38 | 15.23 ± 2.31 |
| Ex B² | 9% | 5.37 ± 0.28 | 21.80 ± 1.64 |
| Ex 2 | 0% | 5.48 ± 0.61 | 21.50 ± 2.85 |
| Ex 3 | 9% | 3.80 ± 0.48 | 12.27 ± 0.62 |
| Ex 4 | 20% | 2.70 ± 0.18 | 10.90 ± 0.29 |

¹Example A: SENSODYNE ® PARODONTAX ™ (LOT: 14042610 EXP24APRIL17); containing around 67% w/w Baking Soda.
²Example B: CREST ® Baking Soda & Peroxide Whitening (LOT: 632232 EXPOCT19); containing around 9% w/w Baking Soda.

Table 3 looks at the simultaneous bactericidal effect of these sodium bicarbonate treatments by the previously described oral care compositions Examples 1-4 and Examples A and B. The fluorescence intensity percentage of live bacteria within in situ plaque biofilm is provided for the same treatments described above. The procedures as previously described are used. Briefly, the biofilm is treated with the subject oral care compositions first, and then the treated biofilm is labeled with the L7012 LIVE/DEAD® dye. Using software, the mean fluorescence intensities of green pixels (staining alive bacteria) and red pixels (staining dead bacteria) are given, then the fluorescence intensity percentage of green pixels is calculated. The lower the mean bacterial vitality percentage, the more effective is the composition.

Still referencing Table 3, Ex 1 (PBS) is used as the negative control. Accordingly, Ex 1 is the least effective (compared to the other compositions) in the mean bacterial vitality percentage (at 91%). Ex A is SENSODYNE® PARODONTAX™ toothpaste (“PARODONTAX”, LOT #14042610, containing 67 wt % sodium bicarbonate), a commercially available toothpaste composition. Ex A shows significantly reduced mean bacterial vitality percentage compared to the negative control (i.e., Ex 1). Notable, however, there is no significant difference between Ex A and Ex 3. Ex 3 is an inventive composition that contains 9 percent, by weight of the composition, of sodium carbonate. This is considerably less than the 67 wt % of Ex A. Ex B is a CREST® Baking Soda & Peroxide Whitening (LOT: 632232 EXPOCT19); containing around 9 wt % of Baking Soda. Ex B also shows significantly reduced mean bacterial vitality percentage compared to the negative control of Ex 1. Notably, however, inventive composition Ex3, taking into account the error margin, is significantly more effective than Ex B. Ex. 4 is the most effective in providing a reduced mean bacterial vitality percentage at 43%.

TABLE 3
Data is provided on Bacterial vitality for various comparative
and inventive dentifrice products/formulations.

| | Weight Percentage of | Bacterial Vitality (%) |
|---|---|---|
| Product¹ | sodium carbonate (wt %) | (Mean) |
| Ex 1 | 0% | 91% |
| Ex A¹ | 67% | 60% |
| Ex B² | 9% | 64% |
| Ex 2 | 0% | 76% |
| Ex 3 | 9% | 58% |
| Ex 4 | 20% | 43% |

¹Control A: SENSODYNE ® PARODONTAX ™ (LOT: 14042610 EXP24APRIL17); containing around 67% w/w Baking Soda
²Control B: CREST Baking Soda & Peroxide Whitening (LOT: 632232 EXPOCT19); containing around 9% w/w Baking Soda

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A dentifrice composition comprising:
(a) 30% to 55%, by weight of the composition, of water;
(b) 25% to 50%, by weight of the composition, of a calcium-containing abrasive;
(c) 1% to 25%, by weight of the composition, a bicarbonate salt;
(d) 0.0025% to 2%, by weight of the composition, of a fluoride ion source;
(e) 0% to 2%, by weight of the composition, of a humectant, wherein the humectant is selected from sorbitol, glycerol, or a combination thereof; and
wherein said composition has a pH greater than 7.8.

2. The dentifrice composition of claim 1, wherein the bicarbonate salt comprises from 1% to 20%, by weight of the composition.

3. The dentifrice composition of claim 2, wherein the bicarbonate salt comprises from 1% to 15%, by weight of the composition.

4. The dentifrice composition of claim 3, wherein the bicarbonate salt comprises from 1% to 10%, by weight of the composition, and wherein the bicarbonate salt is sodium bicarbonate.

5. The dentifrice composition of claim 1, wherein the water is from 34% to 55%, by weight of the composition.

6. The dentifrice composition of claim 4, wherein the water is from 34% to 55%, by weight of the composition.

7. The dentifrice composition of claim 1, wherein the humectant is from 0% to less than 2%, by weight of the composition.

8. The dentifrice composition of claim 7, wherein the humectant is from 0% to 1.5%, by weight of the composition.

9. The dentifrice composition of claim 1, wherein the humectant is a polyol humectant.

10. The dentifrice composition of claim 8, wherein the humectant is a polyol humectant.

11. The dentifrice composition of claim 8, wherein the composition is free of polyol humectant.

12. The dentifrice composition claim 1, wherein the fluoride ion source is sodium monofluorophosphate.

13. The dentifrice composition of claim 10, wherein the fluoride ion source is sodium monofluorophosphate, and is from 0.2% to 1.5% by weight of the composition.

14. The dentifrice composition of claim 1, wherein the calcium-containing abrasive comprises calcium carbonate, and wherein the calcium carbonate is from 27% to 47%, by weight of the composition.

15. The dentifrice composition of claim 14, wherein the calcium carbonate is from 27% to 37%, by weight of the composition.

16. The dentifrice composition of claim 1, wherein the pH is greater than pH 8.5.

17. The dentifrice composition of claim 16, wherein the pH is greater than pH 8.5 to pH 10.5.

18. The dentifrice composition of claim 1, wherein the composition comprises from 0.1% to 5%, of a polyethylene glycol (“PEG”), by weight of the composition.

19. The dentifrice composition of claim 1, wherein the composition comprises from 0.5% to 4% of a polyethylene glycol (“PEG”), by weight of the composition.

20. The dentifrice composition according claim 18, wherein the dentifrice composition comprises a thickening polymer, wherein the thickening polymer is selected from group consisting of:
(a) 0.01% to 3% of a carboxymethyl cellulose (“CMC”), by weight of the composition;
(b) 0.01% to 2.5% of a linear sulfated polysaccharide, by weight of the composition;
(c) 0.01% to 7%, of a natural gum, by weight of the composition; and
(d) combinations thereof.

21. The dentifrice composition according to claim 1, wherein the composition comprises from 0% to 5% silicate, by weight of the composition.
